(19) United States
(12) Patent Application Publication
(10) Pub. No.: US 2006/0074364 A1
(43) Pub. Date: Apr. 6, 2006

(54) Device to prevent brachial plexus injury during childbirth

(76) Inventors: Steven Klein, Old Westbury, NY (US); Robert Couturier, Stamford, CT (US)

Correspondence Address: ROBERT COUTURIER, 1814 NEWFIELD AVENUE, STAMFORD, CT, 06903, US

(21) Appl. No.: 10/956,686

(22) Filed: Oct. 4, 2004

(51) Int. Cl. A61F 5/00

(52) U.S. Cl. 602/18

(57) ABSTRACT

Shoulder dystocia occurs most frequently during the birth of large infants, where the shoulders remain impacted, and lateral traction on the fetal neck with evulsion of the brachial plexus may cause brachial plexus injury. The idea is to prevent excessive lateral bending of the infant's head, its ensuing stress, and damage to the brachial nerve bundle. This invention consists of a special head brace, which limits the lateral movement of the baby's head with respect to the clavicle, and can be quickly mounted when required by the specific medical situation. Its design in two sections provides a snug fit, regardless of the infant's head size, and permits its installation in the little time available to complete the birth process.

### BACKGROUND OF THE INVENTION

Shoulder dystocia is one the most common serious obstetrical emergencies in the delivery room. Shoulder dystocia occurs when the fetal shoulders do not deliver during the normal birth process. The shoulders remain trapped in the birth canal, often obstructed by the mother's pubic bone and sacrum. Although many factors have been associated with shoulder dystocia, such as large fetal size, diabetes or flat maternal pelvis, most cases occur with no warning. Management of this emergency is possible with recognition of the impaction and institution of special obstetrical maneuvers applied to the fetal head, neck, and shoulders, such as the McRobert's maneuver, suprapubic pressure, Wood's rotation, or delivery of the posterior arm. These techniques are designed to relieve the impacted shoulder and allow for spontaneous delivery of the infant.

However, in the course of these maneuvers, substantial forces are inadvertently applied to the fetal head and neck, thereby increasing the risk of severe stretching and tearing of the brachial nerve bundle controlling shoulder, arm, and hand movement. The damage, called brachial plexus palsy, is the cause of paralysis to the newborn's diaphragm, shoulder, arm and/or hand. Shoulder dystocia occurs in approximately one percent of all births, ten to twenty percent result in brachial plexus injury. The purpose of this device is to prevent excessive lateral bending of the infant's head, and its ensuing traction-stress related damage to the vulnerable nerves in the neck during delivery.

### SUMMARY OF THE INVENTION

The invention described herein consists of a special head-neck brace, which limits the lateral movement of the baby's head with respect to the shoulder/clavicle. The device can be quickly mounted as required by the specific medical situation. Its design, in two adjustable sections, provides a contoured fit, regardless of the infant's head/neck size, and permits its rapid (less than 60 seconds) installation to complete the birth process.

### DESCRIPTION OF THE INVENTION

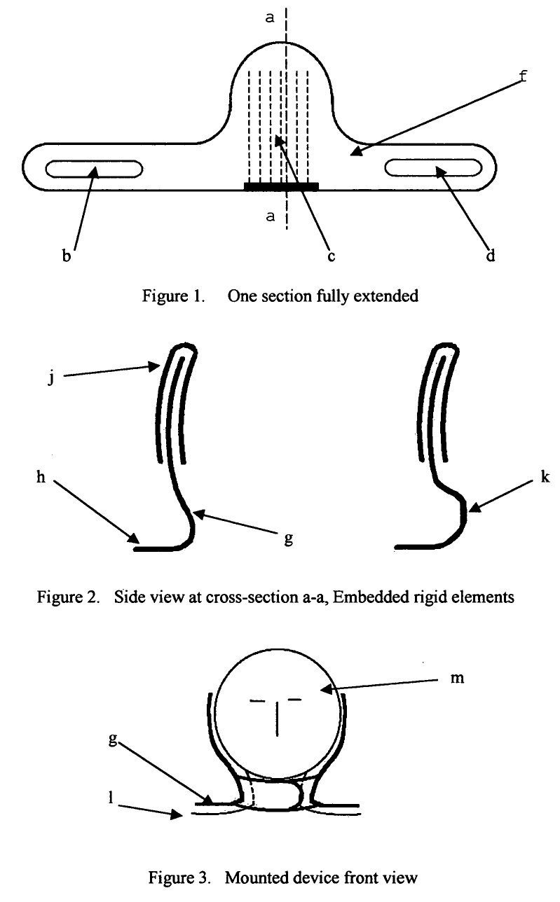

One of the two mirror-image sections is shown on FIG. 1. The flexible strip “f”, made of a non-elastic latex free material, displays three areas. One area “c” contains the rigid elements, which are intended to keep the infant's head in a fixed position relative to its shoulders. The remaining areas are in the form of a strip. Their width is approximately the length of an infant's neck. The ends of these two strips are equipped with a Velcro-type material at the locations “b” and “d”, mounted on the proper side of the strip and with the proper polarity, so that a “b” type locks with a “d” type.

The assembly of two sections is simply performed by first connecting area “b” of section 1 to area “d” of section 2, in such a manner that, when the assembly is placed around the head and jaw of the infant, both areas “c” are covering the ears of the infant. The area “d” of section 1 is then connected to area “b” of section 2, thereby completely and snugly surrounding the neck of the infant. The angled portion of the rigid elements “h”, as shown in FIG. 2, are resting on the infant” clavicle. FIG. 2 also shows a rigid element “g” as viewed in the cross-section a-a of FIG. 1. It displays a curvature, which is designed to provide maximum contact between the device and the infant's head.

In order to achieve the closest fit to the infant's head, the forward rigid element intended to support the infant's mandible is specifically shaped to that effect, as shown in “k” on FIG. 2, while the rearmost element intended to support the occiput is shaped differently to fit that area FIG. 2 also shows the structure of the rigid elements as being made of two parts. The upper part “j” slides over the lower part “g”, allowing for adjustment of the length of the element. The upper portion of the embedding material in FIG. 1, area “c” is left open for access to the tips of the rigid elements during adjustment.

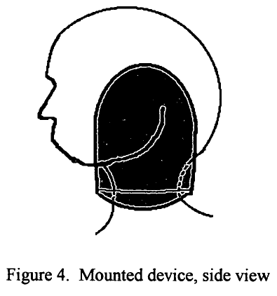

The materials chosen for embedding the rigid elements are supple and flexible while presenting no stretching ability. Their external texture is smooth so as to avoid skin damage to the infant during the birthing maneuvers. FIG. 3 shows the device installed on a head “m”, with the rigid element “g” resting on the clavicle “l”.

In case of emergency obstetric intervention, the device is to be available in the delivery room sterilized and individually wrapped. It is intended for single use.

1- A device consisting of two separate and mirror-image sections, capable of being attached to each other via quick-connect elements such as, but not limited to Velcro material, containing a plurality of rigid elements shaped so as to follow the contour of the sides of the head of an infant, said rigid elements also curved so as to extend at an angle permitting them to simultaneously rest on the clavicular areas below, and the mandible, ear and occiput above.

2- A device as in claim 1, where the rigid elements, which may be produced of suitable metals or plastics, are embedded in a material, the properties of which are flexibility, non-stretchability, external smoothness, and containing no latex nor any known substance causing potentially allergic reaction in the mother.

3- A device as in claim 1, where said rigid elements are adjustable in length.
